(19) United States
(12) Patent Application Publication
(10) Pub. No.: US 2023/0270893 A1
(43) Pub. Date: Aug. 31, 2023

(54) GALLIUM-LABELED GASTRIN ANALOGUE AND USE IN A METHOD OF IMAGING CCKB-RECEPTOR-POSITIVE TUMORS OR CANCERS

(71) Applicant: DEBIOPHARM INTERNATIONAL S.A., Lausanne (CH)

(72) Inventors: Frederic Levy, Lausanne (CH); Antoine Attinger, Colombier (CH)

(21) Appl. No.: 18/040,024

(22) PCT Filed: Jul. 30, 2021
(86) PCT No.: PCT/EP2021/071419

§ 371 Date: Jan. 31, 2023

(30) Foreign Application Priority Data
Jul. 31, 2020 (EP) 20189037.3

(51) Int. Cl. A61K 51/08

(52) U.S. Cl. CPC A61K 51/088

(57) ABSTRACT

A 68Ga-labeled gastrin analogue and its use in peptide receptor radionuclide diagnostic applications is provided. In particular, a 68Ga-labeled minigastrin analogue for use in a method of imaging one or more cholecystokinin B receptor positive diseases selected from small-cell lung cancer, extrapulmonary small-cell carcinoma and medullary thyroid cancer is provided. A kit including the gastrin analogue is also provided.

The present invention relates to a Gallium 68-labeled gastrin analogue and its use in peptide receptor radionuclide diagnostic applications. In particular, the present invention relates to a Gallium 68-labeled minigastrin analogue for use in a method of imaging CCKB-receptor-positive cancers or tumors, which enables improved imaging of specific cancer or tumor tissues and a kit providing the same.

### BACKGROUND OF THE INVENTION

G-protein-coupled receptors (GPCRs) constitute a superfamily of membrane proteins whose function is to transduce a chemical signal across the cell membrane. When a ligand binds to a GPCR, it causes a conformational change allowing the GPCR to activate and release associated G proteins, which subsequently trigger signal transduction pathways.

Overexpression of G-protein-coupled receptors (GPCRs) that selectively bind their peptide ligands allow the development of peptide receptor radionuclide therapy (PRRT) for human cancers (Lappano et al. Nat Rev Drug Discov. 2011, 10(1), 47-60). One of the most important goal of PRRT is to achieve high tumor uptake of radiolabeled ligands. Therefore, strategies to increase the uptake of radiopharmaceuticals in tumors or cancer tissue while sparing healthy organs from cytotoxic side effects have been considered.

GPCRs targeted by agonistic ligand-based therapeutics undergo conformational changes, which lead to the exchange of GDP for GTP on the G-protein alpha subunit (Gα). Subsequent dissociation of the Gα and Gβγ subunits from the receptor results in activation of various kinase signaling pathways involving protein kinases A and C (PKA; PKC) as well as phosphoinositide 3-kinase (PI3K) and mitogen activated protein kinases (MAPKs) (O'Hayre et al. Curr Opin Cell Biol. 2014, 27, 126-135). Subsequently, activated GPCRs undergo desensitization via an arrestin-mediated internalization process, whereby GPCRs can be trafficked to lysosomes for degradation, or to endosomes for their recycling back to the cell surface (Rajagopal et al. Cell Signal. 2018, 41, 9-16). This internalization process enables the delivery of ligand-conjugated radioactive nuclides into target cells, e.g. cancer cells.

Medullary thyroid cancer (MTC) is a neuroendocrine tumor derived from calcitonin-producing C cells. Accounting for 3-5% of all thyroid cancers, MTC is a relatively rare cancer entity (Hadoux et al. Lancet Diabetes Endocrinol. 2016, 4(1), 64-71). Unfortunately, responses to conventional chemotherapy (usually doxorubicin alone or in combination with cisplatin) are only transient and benefit is limited to a small number of patients. In addition, MTC cells do not accumulate iodine and thus, do not respond to radioactive iodine treatment (Verburg et al. Methods. 201, 55(3), 230-237). Currently, MTC accounts for 14% of all thyroid cancer-related deaths, indicating the need for better treatments especially in metastasized patients (Roman et al. Cancer. 2006, 107(9), 2134-2142).

Small-cell lung cancer (SCLC) is a highly malignant cancer that most commonly arises in the lung. It usually presents large, rapidly developing lesions arising from the centrally located tracheobronchial airways and invading the mediastinum. For one third of patients diagnosed with SCLC at a limited-stage of disease, chemoradiotherapy leads to a cure rate of approximately 25%. On the other hand, both extensive-stage and relapsed SCLC are often considered incurable and available treatments, e.g. chemotherapy, are usually administered with a palliative intent. The prognosis of patients with relapsed SCLC remains dismal, with a median overall survival of about 6 months (Travis et al. J Thorac Oncol. 2015, 10(9), 1243-1260).

Extrapulmonary small-cell carcinoma (EPSCC) refers to small-cell carcinomas arising outside the lungs. They most commonly develop in the gastrointestinal and genitourinary systems. EPSCCs are rare neoplasms constituting only 2.5% to 5.0% of all small-cell carcinoma cases and 0.1% to 0.4% of all cancers. EPSCC has an aggressive natural history characterized by rapid local progression, early widespread metastases, and recurrence following treatment. The prognosis of patients diagnosed with EPSCC is relatively poor despite chemotherapy, with median survival ranging from 3 to 27 months and overall 5-year survival rates around 13% (Nakazawa et al. Oncol Lett. 2012, 4(4), 617-620).

High expression of cholecystokinin B receptor (CCKBR, sometimes also referred to as CCK2R), which belongs to the GPCR family, has been validated in some particular forms of cancer including medullary thyroid cancer (MTC), gliomas, as well as colon cancer and ovarian cancer (Reubi et al. Cancer Res 1997, 57(7), 1377-1386). Furthermore, the small peptide hormone minigastrin is known to bind CCKBR with high affinity. Therefore, previous studies have suggested using specific radiolabeled (mini)gastrin-derived peptides such as PP-F11N for targeted peptide receptor radionuclide therapy (PRRT), see WO 2015/067473 A1 (Paul Scherrer Institut).

However, there is continued demand for radiochemicals that can be effectively used in a method of imaging a CCKBR positive tumor or cancer, in particular highly malignant forms such as small-cell lung cancer and extrapulmonary small-cell carcinoma.

In view of the foregoing, it is an object of the present invention to provide a novel radiochemical and its use in a method of imaging specific CCKBR positive tumor or cancer types.

### SUMMARY OF THE PRESENT INVENTION

The present invention provides a novel radiolabeled minigastrin analogue and its use in a method of imaging a CCKBR positive tumors and cancer types.

The present inventors have found that administering an effective dose of a specific minigastrin analog labeled with ⁶⁸Ga to human patients leads to sufficient uptake of the radiolabeled gastrin analog in the tumor or cancer cells, in particular tumor or cancer selected from small-cell lung cancer (SCLC), extrapulmonary small-cell carcinoma (EPSCC) and medullary thyroid cancer (MTC). This finding can be used for the imaging of the cancer/tumor cells or tissues, while unspecific accumulation in healthy cells or tissues can be sufficiently prevented.

The present invention includes among others the following embodiments (“Items”):

(Item 1) Labeled gastrin analogue represented by the following formula (1):

⁶⁸Ga-Y-DGlu-DGlu-DGlu-DGlu-DGlu-DGlu-Ala-Tyr-Gly-Trp-Nle-Asp-Phe-NH₂ (1)
wherein Y is a moiety chelating ⁶⁸Ga.

(Item 2) Labeled gastrin analogue according to item 1 wherein Y is 1,4,7,10-tetraazacyclododecane-1,4,7,10-tetraacetic acid (DOTA), 1,4,7-triazacyclononane-1,4,7-triacetic acid (NOTA) or 1-(1,3-carboxypropyl)-4,7-carboxymethyl-1,4,7-tetraacetic acid (NODAGA).

(Item 3) Labeled gastrin analogue according to item 2 wherein Y is DOTA.

(Item 4) Labeled gastrin analogue according to any of items 1 to 3 for use in a diagnostic method comprising the steps of (i) administering the labeled gastrin analogue to a human patient which is to be diagnosed as to whether he suffers from a cholecystokinin B receptor (CCKBR) positive cancer or tumor and (ii) obtaining an image of the body parts or tissue to be examined,
wherein the cancer or tumor is selected from small-cell lung cancer (SCLC), extrapulmonary small-cell carcinoma (EPSCC) and medullary thyroid cancer (MTC).

(Item 5) Labeled gastrin analogue for the use according to item 4, wherein the administration of the labeled gastrin analogue is used to identify patients that would benefit from a treatment with a compound of the following formula (2):

¹⁷⁷Lu-DOTA-DGlu-DGlu-DGlu-DGlu-DGlu-DGlu-Ala-Tyr-Gly-Trp-Nle-Asp-Phe-NH₂ (2),
wherein DOTA is 1,4,7,10-tetraazacyclododecane-1,4,7,10-tetraacetic acid.

(Item 6) A method for obtaining an image of a patient, or body parts or tissue of said patient, said method comprising administering to a patient the labeled gastrin analogue defined in any of items 1 to 3.

(Item 7) Method according to item 6, said method being PET (Positron Emission Tomography).

(Item 8) Method according to item 6 or 7 wherein the image prepared is that of a cancer or tumor selected from small-cell lung cancer (SCLC), extrapulmonary small-cell carcinoma (EPSCC) and medullary thyroid cancer (MTC).

(Item 9) A kit comprising
(a) a gastrin analogue represented by the following formula (3):

Y-DGlu-DGlu-DGlu-DGlu-DGlu-DGlu-Ala-Tyr-Gly-Trp-Nle-Asp-Phe-NH₂ (3)
wherein, Y is a moiety capable of chelating ⁶⁸Ga,
(b) excipients selected from a solvent, such as water, and one or more auxiliary substances, such as sodium acetate buffer, mannitol and ascorbic acid, said excipients being capable of dissolving the gastrin analogue and providing a solution capable of being labelled with ⁶⁸Ga in a chelation step, wherein these excipients may be provided together or separately.

(Item 10) Use of the kit of item 9 together with a ⁶⁸Ga generator to produce the labeled gastrin analogue represented by the formula (1) as defined in any of items 1 to 3.

### DESCRIPTION OF FIGURES

FIG. 1 shows ex vivo biodistribution studies conducted with female (FIG. 1A) and male (FIG. 1B) naïve adult CD1 mice (immunocompetent) which were injected with [⁶⁸Ga]Ga-PPF11N.

### DETAILED DESCRIPTION OF THE PRESENT INVENTION

The present invention provides a novel minigastrin analogue labeled with ⁶⁸Ga and an imaging method using the same. The imaging method can be used for the diagnosis of specific CCKBR positive tumors or cancer types including small-cell lung cancer (SCLC), extrapulmonary small-cell carcinoma (EPSCC) and medullary thyroid cancer (MTC).

The biodistribution studies conducted with ⁶⁸Ga-labeled minigastrin of formula (1) show that the minigastrin binds specifically to CCK2R receptors while the unspecific accumulation in essentially all healthy tissues and/or organs remains at an acceptable low level. Only in the kidneys was a relatively high exposure observed. Since however the peptide blocking experiments were unable to reduce kidney exposure, kidney exposure is CCK2R-target independent. This demonstrates that the kidney exposure is most likely caused by elimination and/or renal uptake.

Moreover, the CCKBR prevalence studies reported in the experimental section indicate that the use of the ⁶⁸Ga-labeled minigastrin will be effective in the diagnosis of specific tumor and cancer types while other cancer tissue isolated from human patients did not show the required prevalence (i.e. pancreatic adenocarcinoma (PDAC) or gastric cancer (GC)).

### 1. Definitions and Embodiments

The expression “gastrin analogue” as used herein refers to a class of compounds (peptides) structurally related to the endogenous peptide hormone gastrin, which can bind to the CCKBR. Gastrin is a linear peptide hormone produced by G cells of the duodenum and in the pyloric antrum of the stomach. It is secreted into the bloodstream. The encoded polypeptide is pre-progastrin, which is cleaved by enzymes in posttranslational modification to produce progastrin and then gastrin in various forms, including primarily big-gastrin (G-34), little gastrin (G-17), and minigastrin (Leu-Glu-Glu-Glu-Glu-Glu-Ala-Tyr-Gly-Trp-Met-Asp-Phe-NH₂). CCK is a peptide hormone structurally related to gastrin in that both compounds share five C-terminal amino acids i.e. Gly-Trp-Met-Asp-Phe-NH₂ (wherein Met can be replaced by an amino acid isosteric with Met such as norleucine). CCK exists naturally in several forms including e.g. CCK8 (Asp-Tyr-Met-Gly-Trp-Met-Asp-Phe-NH₂). Gastrin and peptide hormones related thereto typically contain the C-terminal amino acid motif “Gly-Trp-Met-Asp-Phe-NH₂”, which enables their binding to CCKBR.

The pharmacological activity of a given gastrin analogue towards CCKBR can be determined by measuring the intracellular increase of calcitonin level in gastrin analogue-stimulated cells as described by Bläker et al. (Regulatory Peptides 2004, 118, 111-117).

The term “cancer” as used herein means the pathological condition in mammalian tissues that is characterized by abnormal cell growth to form malignant tumors, which may have the potential to invade or spread to other tissues or parts of the body to form “secondary” tumors known as metastases. A tumor comprises one or more cancer cells. The expression “CCKBR positive cancer or tumor” as used herein refers to cancers or tumors that are characterized by overexpression of the CCKBR on the cell surface (Reubi et al. Cancer Res. 1997, 57(7), 1377-1386). Examples of CCKBR positive cancer or tumors include MTC, gliomas, small-cell lung cancer, astrocytomas, colon cancer, ovarian cancer and breast cancer. In one preferred embodiment, the expression “CCKBR positive cancer or tumor” as used herein refers to small-cell lung cancer (SCLC) or extrapulmonary small-cell carcinoma (EPSCC).

The expression “tumor uptake” (of radiopharmaceuticals) refers to the biological process in which molecules (e.g. the minigastrin of formula (1)) are taken up by tumor (cancer) cells. Tumor uptake includes tumor cell uptake of molecules (e.g. the radiolabeled gastrin analogue) and/or the retention thereof in the tumor microenvironment. As a result, the molecules (e.g. the radiolabeled gastrin analogue) can be present inside the tumor (cancer) cell, at the cell membrane (e.g. accumulated on the cell membrane) and/or within the tumor microenvironment. The radioactivity emitted can thereby be used to visualize (image) the tumor.

The expression “effective dose” (or “effective amount”) as used herein refers to the “imaging dose” (i.e. total dose of radioactivity administered to the patient to carry out imaging such as SPECT or PET CT imaging of the tumor tissues).

The effective dose can be determined by a physician based on dosimetry. The effective dose and frequency of dosage for any particular subject/patient can vary and depends on a variety of factors including the patient's age, body weight, general health, sex, diet, mode and time of administration, rate of excretion, the severity of the disease, and the individual undergoing therapy. These factors are considered by the physician when determining the effective dose.

Where the present description refers to “preferred” embodiments/features, combinations of these “preferred” embodiments/features shall also be deemed as disclosed as long as this combination of “preferred” embodiments/features is technically meaningful.

Hereinafter, in the present description of the invention and the claims, the use of the terms “containing” and “comprising” is to be understood such that additional unmentioned elements may be present in addition to the mentioned elements. However, these terms should also be understood as disclosing, as a more restricted embodiment, the term “consisting of” as well, such that no additional unmentioned elements may be present, as long as this is technically meaningful.

Unless the context dictates otherwise and/or alternative meanings are explicitly provided herein, all terms are intended to have meanings generally accepted in the art, as reflected by IUPAC Gold Book (status of 1, Nov. 2017), or the Dictionary of Chemistry, Oxford, 6^{th} Ed.

### 2. Gallium 68 Labeled Minigastrin Analogue

The labeled gastrin analogue of the present invention is represented by the formula (1):

⁶⁸Ga-Y-(DGlu)₆-Ala-Tyr-Gly-Trp-Nle-Asp-Phe-NH₂ (1)

wherein Y is a moiety chelating Gallium 68 (⁶⁸Ga). Preferably the chelator moiety is covalently attached to the N-terminus of the peptide chain via one functional group, e.g. carboxyl group. If Y is selected 1,4,7,10-tetraatacyclododecane-1,4,7,10-tetraacetic acid (DOTA), the compound of formula (1) is known as PP-F11N.

Unless specified otherwise or dictated otherwise by the context, all connections between adjacent amino acid groups are formed by peptide (amide) bonds. The peptides described herein are listed in the conventional amino- to carboxy-direction from left to right.

The expression “moiety chelating Gallium 68 (⁶⁸Ga)” as used herein refers to a moiety (e.g. DOTA) that can donate electrons to Gallium 68 (⁶⁸Ga) to form a coordination complex therewith, e.g. by forming at least one coordinate covalent bond (dipolar bond) therewith. It has been described in the art that e.g. DOTA is capable of coordinating a radionuclide such as ⁶⁸Ga via carboxylate and amino groups (donor groups) thus forming complexes having high stability.

Examples of moieties chelating Gallium 68 (⁶⁸Ga) include but are not limited to diethylenetriaminepentaacetic acid (DTPA), desferoxamine (DFO), 1-(1,3-carboxypropyl)-4,7-carboxymethyl-1,4,7-tetraacetic acid (NODAGA), 1,4,7,10-tetraazacyclododecane-1-glutaric acid-4,7,10-triacetic acid (DOTAGA), triazacyclononane-1,4-diyl)diacetate (NO2A), 1,4,7,10-tetraatacyclododecane-1,4,7,10-tetraacetic acid (DOTA), 1,4,7-triazacyclononane-1,4,7-triacetic acid (NOTA), ethylenediaminetetraacetic acid (EDTA), ethylenediaminediacetic acid, triethylenetetraminehexaacetic acid (TTNA), 1,4,8,11-tetraazacyclotetradecane (CYCLAM), 1,4,8,11-tetraazacyclotetradecane-1,4,8,11-tetraacetic acid (TETA), 1,4,8,11-tetraazabicyclo[6.6.2]hexadecane-4,11-diaceticacid (CB-TE2A), 2,2′,2″-(1,4,7,10-tetraazacyclododecane-1,4,7-triyl)triacetamide (DO3AM), 1,4,7,10-tetraazacyclododecane-1,7-diacetic acid (DO2A), 1,5,9-triazacyclododecane (TACD), (3a1s,5a1s)-dodecahydro-3a,5a,8a,10a-tetraazapyrene (cis-glyoxal-cyclam), 1,4,7-triazacyclononane (TACN), 1,4,7,10-tetraazacyclododecane (cyclen), tri(hydroxypyridinone) (THP), 3-(((4,7-bis((hydroxy(hydroxymethyl)phosphoryl)methyl)-1,4,7-triazonan-1-yl)methyl)(hydroxy)phosphoryl)propanoic acid (NOPO), 3,6,9,15-tetraazabicyclo[9.3.1]pentadeca-1(15), 11,13-triene-3,6,9-triacetic acid (PCTA), 2,2′,2″,2′″-(1,4,7,10-tetraazacyclotridecane-1,4,7,10-tetrayl)tetraacetic acid (TRITA), 2,2′,2″,2′″-(1,4,7,10-tetraazacyclotridecane-1,4,7,10-tetrayl)tetraacetamide (TRITAM), 2,2′,2″-(1,4,7,10-tetraazacyclotridecane-1,4,7-triyl)triacetamide (TRITRAM), trans-N-dimethyl-cyclam, 2,2′,2″-(1,4,7-triazacyclononane-1,4,7-triyl)triacetamide (NOTAM), oxocyclam, dioxocyclam, 1,7-dioxa-4,10-diazacyclododecane, cross-bridged-cyclam (CB-cyclam), triazacyclononane phosphinate (TRAP), dipyridoxyl diphosphate (DPDP), meso-tetra-(4-sulfanotophenyl)porphine (TPPS₄), ethylenebishydroxyphenylglycine (EHPG), hexamethylenediaminetetraacetic acid, dimethylphosphinomethane (DMPE), methylenediphosphoric acid, dimercaptosuccinic acid (DMPA), and derivatives thereof.

In one embodiment, Y is an N-containing macrocycle to which one or more carboxy-bearing side chains (e.g. 3 or 4) have been attached. The macrocycle includes preferably 3 or 4 N atoms and the preferred number of rings atoms (N and C) is at least 12 and preferably not more than 20 (e.g. 12 to 16). Examples of the carboxy-bearing side chains include carboxymethyl (acetic acid), propanoic acid, carboxypropyl or glutaric acid.

Moiety Y is more preferably 1,4,7,10-tetraazacyclododecane-1,4,7,10-tetraacetic acid (DOTA), 1,4,7-triazacyclononane-1,4,7-triacetic acid (NOTA) or 1,4,7-triazacyclononane,1-glutaric acid-4,7-acetic acid (NODAGA) and most preferably Y is DOTA.

The gastrin analogue PP-F11N can be synthesized relying on standard Fmoc-based solid-phase peptide synthesis (SPPS), including on-resin peptide coupling and convergent strategies. The general strategies and methodology which can be used for preparing and radiolabeling the gastrin analogue of the present invention are well known to the skilled person and further illustrated below in the examples.

### 3. Use of Compound in Method of Imaging Diseases and Imaging Method

The compound of the present invention can be suitably used in methods of imaging a CCKBR positive cancer or tumor (for instance SCLC, EPSCC or MTR) in which cancer or tumor cells are visualized by imaging techniques such as known computer tomography techniques such as PET (PET=Positron Emission Tomography); for a review of this technique and its application see e.g. Shankar Vallabhajosula (ed.), Molecular Imaging, Radiopharmaceuticals for PET and SPECT, Springer Verlag or Lucia Martiniova et al., Gallium-68 in Medical Imaging, Current Radiopharmaceuticals, 2016, 9, 187-207. The radiolabeled gastrin analogue can thus be used for diagnosing the progression and/or state of the CCKBR positive cancer or tumor.

The present invention also relates to a diagnostic method comprising the steps of (i) administering the labeled gastrin analogue of formula (1) to a human patient which is to be diagnosed as to whether he suffers from a cholecystokinin B receptor (CCKBR) positive cancer or tumor and (ii) obtaining an image of the body parts (e.g. lung/s or thyroid gland/s) or tissue (e.g. lung or thyroid tissue) to be examined, wherein the cancer or tumor is selected from small-cell lung cancer (SCLC), extrapulmonary small-cell carcinoma (EPSCC) and medullary thyroid cancer (MTC).

In one embodiment of this method the labeled gastrin analogue is used to identify patients that would benefit from a treatment with a compound of the following formula (2):

¹⁷⁷Lu-DOTA-DGlu-DGlu-DGlu-DGlu-DGlu-DGlu-Ala-Tyr-Gly-Trp-Nle-Asp-Phe-NH₂ (2) (=¹⁷⁷Lu-DOTA-PPF11N),
wherein DOTA is 1,4,7,10-tetraazacyclododecane-1,4,7,10-tetraacetic acid.

The present inventors have also found that the ⁶⁸Ga-DOTA-PPF11N biodistribution broadly resembles that of ¹⁷⁷Lu-DOTA-PPF11N. Therefore, the imaging with ⁶⁸Ga-PPF11N might be particularly suitable to pre-select patients that are expected to benefit from a ¹⁷⁷Lu-PPF11N treatment. This is very beneficial since, otherwise, it would be necessary to administer ¹⁷⁷Lu-PPF11N to the entire group of patients for therapeutic and diagnostic purposes. However, even at low doses of ¹⁷⁷Lu-PPF11N the energy of the emitted radiation is so strong that undesired side effects can easily occur. The pre-selection of patients by means of the above method therefore significantly increases the efficacy of any kind of ¹⁷⁷Lu-PPF11N treatment while minimizing side effects.

The present invention also relates to a method for obtaining an image of a patient, or body parts (e.g. lung/s or thyroid gland/s) or tissue (e.g. lung or thyroid tissue) of said patient, said method comprising administering to a patient the ⁶⁸Ga-labeled gastrin analogue described herein. This method is preferably PET (Positron Emission Tomography). In one preferred embodiment, this method is used to obtain an image of a cancer or tumor selected from small-cell lung cancer (SCLC), extrapulmonary small-cell carcinoma (EPSCC) and medullary thyroid cancer (MTC).

Visualization is achieved by recording the energy and location of the radiation emitted by ⁶⁸Ga (“tracer”), this information then being used by a computer program to reconstruct three-dimensional (3D) images of tracer concentration within the body.

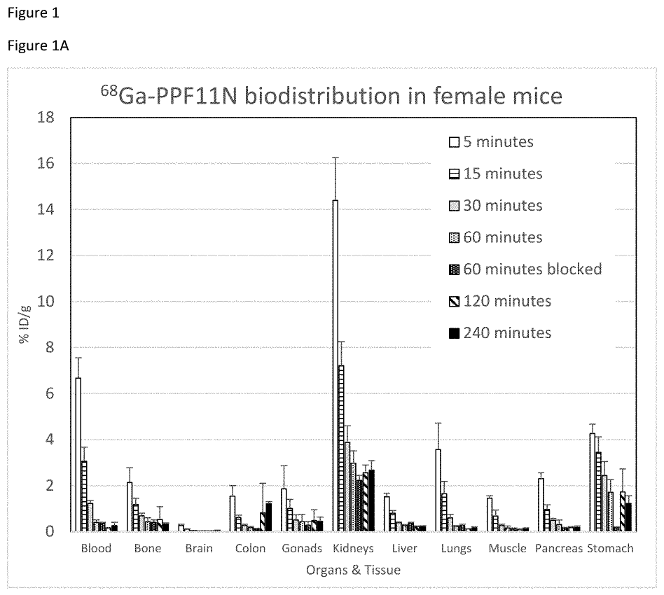

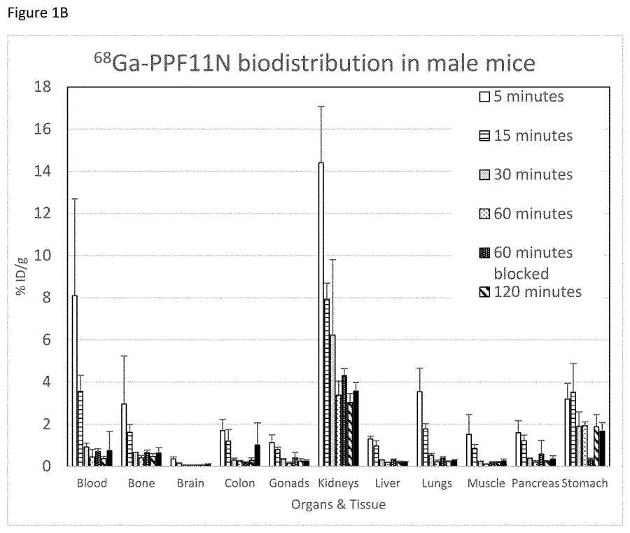

The convenient half-life of ⁶⁸Ga (T1/2=68 min) provides sufficient radioactivity for various PET imaging applications. ⁶⁸Ga decays 87.94% through positron emission with a maximum energy of 1.9 MeV, mean 0.89 MeV (FIG. 1). The ⁶⁸Ga³⁺ cation can form stable complexes with many ligands containing oxygen and nitrogen as donor atoms. This makes ⁶⁸Ga suitable for complexation with chelators and various macromolecules, allowing for kit development.

In modern PET computed tomography scanners, PET images are often reconstructed with the aid of a computed tomography scan performed on the patient during or shortly after the administration of the tracer, in the same device.

PET images obtained with ⁶⁸Ga show a very high resolution, typically much higher than that achievable by SPECT (Single Photon Emission Computed Tomography). ⁶⁸Ga can also be used in diagnostic method utilizing the compound of formula (1) as tracer. SPECT is similar to PET in its use of radioactive tracer material. In contrast to PET, the tracers used in SPECT emit gamma radiation that is measured directly, whereas PET tracers such as ⁶⁸Ga emit positrons that annihilate with electrons up to a few millimeters away, causing two gamma photons to be emitted in opposite directions. A PET scanner detects these emissions “coincident” in time, which provides more radiation event localization information and, thus, higher spatial resolution images than SPECT (which has about 1 cm resolution).

For imaging purposes, the effective dose of the compound to be administered to the patient (imaging dose) can preferably range from 0.5 to 4 MBq/Kg/person, for example 1 to 3 MBq/Kg/person, or 1.5 to 2.5 MBq/Kg/person, e.g. 2 MBq/Kg/person.

Any known ⁶⁸Ga generator can be used to make ⁶⁸Ga, e.g. a ⁶⁸Ga generator as sold by Eckert and Ziegler or IRE. The produced ⁶⁸Ga can then be mixed with the compound of formula (3), see below item 4, such as PP-F11N, and water (sterile metal free water) and heated, e.g. at 80-100° C., e.g. 90-95° C. to form a solution comprising compound of formula (1), such as ⁶⁸Ga-PPF11N, this product may then be quality controlled before being delivered to the patient for administration.

The quantity of the compound of formula (1), such as ⁶⁸Ga-PPF11N, for administration can then be calculated by a medical practitioner to equate to an effective dose of the compound of formula (1), such as ⁶⁸Ga-PPF11 N. This quantity or amount for administration will dependent on the concentration of compound of formula (1), such as ⁶⁸Ga-PPF11N calculated to be in the composition e.g. taking into consideration time from generation of the ⁶⁸Ga and its half-life.

Administration is preferably intravenously using a non-metallic syringe.

### 4. Kit of Parts

The present invention also relates to a kit which can be conveniently used to prepare shortly before its administration to a patient. This kit comprises
(a) a gastrin analogue represented by the following formula (3):

Y-DGlu-DGlu-DGlu-DGlu-DGlu-DGlu-Ala-Tyr-Gly-Trp-Nle-Asp-Phe-NH₂ (3)
wherein, Y is a moiety capable of chelating ⁶⁸Ga, and
(b) excipients selected from a solvent, such as water, and one or more auxiliary substances, said excipients being capable of dissolving the gastrin analogue and providing a solution capable of being labelled with ⁶⁸Ga in a chelation step, wherein these excipients may be provided together or separately.

As a solvent, preferably metal-free water (e.g. “traceselect” water) is used. The auxiliary substances can be selected from common excipients which include, but are not limited to pharmaceutically acceptable buffering compounds, sugars, stabilisers and/or antioxidants such as ascorbic acid or gentisic acid.

In one embodiment, the compound of formula (3), e.g. PP-F11N, is provided in one part of the kit in dry form together a sugar, such as mannitol, and an antioxidant such as ascorbic acid.

In this embodiment, the buffer substance, such as sodium acetate buffer, is provided in a different, e.g. second, part of the kit, preferably in dry form, and dissolved in pharmaceutical grade water, in particular metal-free water.

Sterile metal-free water may be provided in a third part of the kit.

The resulting aqueous buffer solution is then used to dissolve the compound of formula (3), e.g. PP-F11N, together with the other auxiliary substances provided in the first part of the kit.

⁶⁸Ga provided by gallium-68 generator is added to the buffered solution to chelate the ⁶⁸Ga, optionally under heating as described before. Heating may for instance be carried out over a time period of 5 min to 1 h, e.g. 10 min to 30 min, such as 20 min.

In this manner, the kit of the invention can be used together with a commercially available ⁶⁸Ga generator to produce the labeled gastrin analogue represented by the following formula (1).

Usually, the kit will also comprise Instructions detailing how to chelate ⁶⁸Ga to the gastrin analogue to form a labeled gastrin analogue of the invention and/or instructions on how to use such labeled gastrin analogue in a method of diagnosis or a Method of obtaining an image of a patient as claimed.

### 5. Examples

### List of Abbreviations

BSA: bovine serum albumin
DIEA: diisopropylethylamine
DMF: dimethyl formamide
EGTA: ethylene glycol-bis(3-aminoethyl ether)-N,N,N′,N′-tetraacetic acid
ESI: electron spray ionization
HATU: 1-[Bis(dimethylamino)methylene]-1H-1,2,3-triazolo[4,5-b]pyridinium 3-oxide hexafluorophosphate
HBTU: 3-[Bis(dimethylamino)methyliumyl]-3H-benzotriazol-1-oxide hexafluorophosphate
HPLC: high-performance liquid chromatography
SQD: single quadrupole detection
SPECT: single-photon emission computed tomography
SPPS: solid-phase peptide synthesis
TFA: trifluoroacetic acid
TIS: triisopropylsilane
Tris: tris(hydroxyethyl)aminomethane
UPLC: ultra-performance liquid chromatography

The following materials and methods were used to evaluate the compound of the present invention.

Tissue Acquisition and Preparation

Tissue (fresh frozen blocks) isolated from twenty SCLC, four MTC, twenty GC and twenty PDAC patients were acquired from a Tissue Biobank supplier. Tissues were allowed to equilibrate for at least 1 h in the cryotome chamber of a Leica 3050 before sectioning at −18° C. (chamber temperature) and at a thickness of 20 μm.

Autoradiography

The following buffers were prepared freshly before each assay

TABLE 1
Autoradiography buffers

| | |
|---|---|
| Pre-incubation buffer (Pre-IB) | 0.05M Tris |
| | 130 mM NaCl |
| | 4.7 mM KCl |
| | 1 mM EGTA |
| | 0.5% BSA |
| | 5 mM MgCl₂ |
| | pH 7.4 |
| Incubation buffer (IB) | Pre-IB without BSA |
| | 0.025% Bacitracin |
| | 25 mg Dithiothreitol |
| | 2 μg/ml Chymostatin |
| | 4 μg/ml Leupeptin |
| | pH 6.5 |
| Wash buffer 1 (WB1) | Pre-IB with 0.5% BSA |
| | pH 7.4 |
| Wash buffer 2 (WB2) | Pre-IB without BSA |
| | pH 7.4 |

Autoradiography Protocol:
The samples (tumor sections) were first dried for at least 5 min using a cold fan to increase tissue adsorption to the slides. Incubation with Pre-IB to reduce potential occupation of the CCKBR was followed by another drying step for 10 min. Afterwards the samples were incubated with ¹¹¹In-PPF11N in IB. In order to assess non-specific binding, an adjacent section was incubated in tracer solution mixed with 200 nM of unlabeled human gastrin I (available from Bachem, Switzerland). After the procedure, slides were washed 6×15 min in pre-cooled WB1 and 2×5 sec in WB2, before drying the sections for at least 15 min using a cold fan. Sections were apposed to a Biomax MR film in X-ray cassette and films were developed in an automated developing machine.

Signal Quantification:
For signal quantification, a separate standard curve was recorded for every experiment. Autoradiograms were quantitatively analysed using MCID software (InterFocus). The region of interest (ROI) was measured twice, once on the tissue with total tracer binding and once on the sample for non-specific binding. Radioligand binding was considered specific if total binding signal was at least twice as high as the non-specific signal (Reubi et al. Cancer Res. 1997, 57(7), 1377-1386). Because the different tumor sample sections are composed of tumoral tissue and normal adjacent tissue, the colocalization of the radio-signal to the tumoral tissue in the slides was confirmed by a Hematoxylin and Eosin (H&E) staining.

Hematoxylin and Eosin (H & E) Staining:
H&E-staining of sections adjacent to the ones used in autoradiography permits localization of the autoradiographic signal. For H&E-staining, frozen tissue sections were fixated for 10 s in 1:1 acetone-ethanol solution (trichloroacetic acid 1 mol/l). Afterwards, they were hydrated in alcohol series (100; 96; 70; 50% EtOH) followed by a brief rinse in H₂O. Incubation in Mayer's hemalaun solution for 10 min stained the nuclei of cells. After washing in H₂O and dd H₂O, the slides were immersed briefly in hydrochloric acid alcohol. Subsequent 10 min incubation in warm water led to a colour change from red to blue. Counterstaining with eosin solution produces a contrast in acidophilic structures (pink). Slides were washed and dehydrated in an ascending alcohol series (70-100%). The samples were placed into xylene twice and subsequently embedded using Eukitt and glass cover slips.

### Reference Example 1: Preparation of PP-FF11 N

The gastrin analogue described and used herein (PP-F11 N) was prepared by standard Fmoc-based SPPS, including on-resin peptide coupling and convergent strategies using an Activo-P-11 Automated Peptide Synthesizer (Activotec) and a Rink Amide resin (loading: 0.60 mmol/g; Novabiochem).

Coupling reactions for amide bond formation were performed over 30 min at room temperature using 3 eq of Fmoc-amino-acids activated with HBTU (2.9 eq) in the presence of DIEA (6 eq.). Fmoc deprotection was conducted with a solution of 20% piperidine in DMF. Coupling of the N-terminal labeling moiety can be performed over 30 min at room temperature using 3 eq of DOTA tris-t-Bu ester (Novabiochem) activated with HATU (2.9 eq) in the presence of DIEA (6 eq).

The peptide was cleaved from the resin under simultaneous side-chain deprotection by treatment with TFA/TIS/water (95/2.5/2.5, v/v/v) during 60 min. After concentration of the cleavage mixture, the crude peptide was precipitated with cold diethyl ether and centrifugated.

The peptide was purified on a Waters Autopurification HPLC system coupled to SQD mass spectrometer with a XSelect Peptide CSH C18 OBD Prep column (130 Å, 5 μm, 19 mm×150 mm) using solvent system (0.1% TFA in water) and B (0.1% TFA in acetonitrile) at a flow rate of 25 mL/min and a 20-60% gradient of B over 30 min. The appropriate fractions were associated, concentrated and lyophilized. The purity was determined on a Waters Acquity UPLC System coupled to SQD mass spectrometer with CSH C18 column (130 Å, 1.7 μm, 2.1 mm×50 mm) using solvent system A (0.1% TFA in water) and (0.1% TFA in acetonitrile) at a flow rate of 0.6 mL/min and a 5-85% gradient of B over 5 min.

MS-analysis was performed using electrospray ionization (ESI) interface in positive and negative mode.

### Reference Example 2: Indium Radiolabeling of PP-FF11N

To prepare the indium-labeled gastrin analogue used in Example 1 below (¹¹¹In-PP-F11 N), a solution of PP-F11N was added to the radionuclide solution (¹¹¹InCl₃ in 20 mM HCl, available from Curium). Labeling buffer (sodium acetate pH 5.3) was added to a final concentration of 0.1 M buffer. After heating for 25 min at 80° C., the reaction mixture was allowed to cool down for 5 min before adding 1 μl 10 mM DTPA and 1 μl 5% TWEEN-20 per 50 μl. For quality control, the reaction mixture was diluted 1:10 in HPLC sample diluent (0.1% TWEEN-20 in 0.1 M sodium acetate pH 5.3).

Labeling efficiency and radiochemical purity were determined by HPLC using an Agilent Poroshell HPH C18 column (gradient: 5% acetonitrile (ACN) to 70% ACN in 0.1% TFA in water within 15 min; flow rate: 0.5 ml/min). Labeling efficiency and radiochemical purity of ¹¹¹In-PPF11N was greater than 94%.

### Reference Example 3: In Vitro Autoradiography Radioligand Binding Assay of a Radiolabeled Gastrin Analogue (¹¹¹In-PP-FF11N) in SCLC, PDAC, GC, and MTC Tumor Tissues

The capacity of ¹¹¹In-PP-FF11N (prepared as described above) to bind CCKBR in fresh tumor sections collected from patients diagnosed with SCLC, pancreatic adenocarcinoma (PDAC), Medullary thyroid cancer (MTC) or gastric cancer (GC) was evaluated by autoradiography (as described above)

Tissue sections collected from tumors of twenty different patients diagnosed with SCLC were incubated with ¹¹¹In-PP-FF11N and measured. Among the twenty SCLC tissue sections analyzed, four were found to be positive with ¹¹¹In-PP-F11N corresponding to a CCKBR prevalence of 20%.

These results demonstrate that CCKBR is expressed in SCLC tumor tissues, and that the compound of the present invention (e.g. ⁶⁸Ga-PP-F11N) can be used for imaging of SCLC in human patients diagnosed with the disease. Furthermore, these results also demonstrate that the compound of the present invention can be used for treating and/or imaging EPSCC as the clinical features and therapy of this disease are similar to those of SCLC.

Tissue sections collected from tumors of twenty different patients diagnosed with MTC were incubated with ¹¹¹In-PP-FF11N and measured. Among the four MTC tissue sections analyzed, three were found to be positive with ¹¹¹In-PP-F11N corresponding to a CCKBR prevalence of 75%.

These results demonstrate that CCKBR is expressed in MTC tumor tissues, and that the compound of the present invention (e.g. ⁶⁸Ga-PP-F11N) can be used for imaging of MTC in human patients diagnosed with the disease.

Tissue sections collected from tumors of twenty different patients diagnosed with PDAC were incubated with ¹¹¹In-PP-FF11N and analyzed by autoradiography. However, among the twenty PDAC tissue sections analyzed, none was found to be CCKBR positive (no ¹¹¹In-PP-F11N binding). The CCKBR prevalence in the PDAC tumor tissues was thus determined to be 0%.

Tissue sections collected from tumors of twenty different patients diagnosed with GC were incubated with ¹¹¹In-PP-FF11N and analyzed by autoradiography. However, among the twenty GC tissue, in none of them a strict co-localization between the radio-signal and the tumoral tissue was observed. The CCKBR prevalence in the GC tumor tissues was thus determined to be 0%.

The measured CCKBR prevalence is shown in the table below with respect to each tumor type:

TABLE 2
CCKBR prevalence in SCLC, PDAC and GC tumor sections

| Indication | CCKBR prevalence |
|---|---|
| SCLC | 20% |
| PDAC | 0% |
| MTC | 75% |
| GC | 0% |

The above results indicate that the compound of the present invention (e.g. ⁶⁸Ga-PP-F11N) can suitably be used for imaging specific CCKBR positive tumors and cancer types such as SCLC and/or EPSCC due to the sufficiently high CCKBR prevalence in these tumor types. On the other hand, it was found that the CCKBR prevalence in other tumor tissues, i.e. PDAC and GC, was not high enough to enable treatment and/or imaging of these tissues with the compound (due to the lack of specific binding). The latter finding is particularly surprising as PDAC/GC tissues are usually reported in the literature as CCKBR positive tissues.

### Example 1—Preparation of [⁶⁸Ga]Ga-PPF11N

To prepare the Gallium-labelled gastrin analogue (⁶⁸Ga-PPF11N), an eluate of a gallium generator was added to a solution of PPF11N 80 μg, Mannitol 6 mg, ascorbic acid 1 mg and sodium acetate (100 Mg) (pH 3.9). After heating at 95° C. for 20 min, the reaction mixture was allowed to cool down for 10 min. The radiochemical purity was analysed by TLC (thin layer chromatography) 5 μL sample on silica gel plate. Mobile phase 77 g/L solution of ammonium acetate in water, methanol 50:50 VN. Detection with a detector suitable to determine the distribution of radioactivity. No more than 3% of free gallium-68

### Example 2—Ex Vivo Biodistribution Study with [⁶⁸Ga]Ga-PPF11N

### Legend of FIG. 1:

Ex vivo biodistribution study. Female (FIG. 1A) and male (FIG. 1B) naïve adult CD1 mice (immunocompetent) were injected with [⁶⁸Ga]Ga-PPF11N ((0.19±0.04) MBq, 0.04 μg, 1.3 μg/kg). Mice were sacrificed at 5, 15, 30, 60, 120 and 240 min post-injection (N=4 each). A control group of mice of each sex was co-injected with a blocking dose of PPF11N peptide (40 μg, 1.3 mg/kg) and sacrificed at 60 min post-injection. Organs (blood, bone, brain, colon, gonad, kidney, liver, lung, muscle, pancreas, stomach) were weighted and assessed for activity concentration over time. Ex vivo biodistribution in all resected tissues was computed as the percentage of injected dose per gram of tissue (% ID/g).

Materials and Methods:

Immunocompetent CD1 mice (N=28 males and N=28 females on study, N=3 males and N=3 females spare; males 9 weeks old, females 5 weeks old at delivery) were acclimatized for 7 days, inspected daily and weighed on the day of injection. For each sex, mice were randomly allocated to 7 groups (N=4 mice per group) and each group was injected (bolus i.v. route into a laterial tail vein) with [⁶⁸Ga]Ga-PPF11N peptide ((0.19±0.04) MBq in 0.15 mL; (0.039±0.02) μg peptide in non-blocked groups. Following radiolabeling, each product batch was assessed by iTLC and HPLC to ensure the material met the target criteria. For the blocked group, 38±1 μg of peptide was injected. At 5 min, 15 min, 30 min, 1 h, 2 h and 4 h post-injection mice (n=4 per time point for each sex) were sacrificed by cervical dislocation and tissues (blood, bone, brain, colon, gonad, kidney, liver, lung, muscle, pancreas, stomach) were resected, weighed and the radioactivity was counted with a gamma-counter along with calibration standards (dilution series in triplicate). The study design is depicted in the table below.

All doses for injection were prepared the day of injection and 4 radiosynthesis were required for Phase 2. At the end of Phase 2, all spare animals were culled, and carcasses were discarded.

TABLE 3
Summary of treatments schemes

| | | | Number of |
|---|---|---|---|
| | Peptide dose | | mice per |
| Group name (N) | injected (μg) | Cull time points | time point |
| Female Non-Block | 0.04 | 5 min, 15 min, 30 min, | 4 |
| (24) | | 1 h, 2 h, 4 h | |
| Female Block (4) | 40 | 1 h | 4 |
| Male Non-Block | 0.04 | 5 min, 15 min, 30 min, | 4 |
| (24) | | 1 h, 2 h, 4 h | |
| Male Block (4) | 40 | 1 h | 4 |

The activity of each collected tissue was measured in units of counts per min (CPM). Triplicate aliquots of serial dilutions of the radiotracer were also assayed in the gamma counter in order to calculate a conversion factor to units of activity (% ID/g). Measured values were decay corrected and adjusted to account for background radiation

### Results and Conclusions

Results of the ex vivo biodistribution show high similarity between males and females with rapid clearance from all organs except for kidneys and stomach.

Blood concentration reached a plateau at 60 min post-injection (0.40±0.12% ID/g for female group, and 0.46±0.35% ID/g for male group). Most other tissues displayed a similar PK pattern.

Retention of ⁶⁸Ga-PPF11N was observed at 60 min post-injection in kidneys with 2.98±0.53% ID/g for females and 3.38±0.67% ID/g for males, and in stomach with 1.71±0.56% ID/g for females and 1.93±0.18% ID/g for males. The observed retention in both organs was at least one order of magnitude higher compared with muscle with 0.16±0.09% ID/g for females and 0.10±0.03% ID/g for males at 60 min post-injection. The muscle may serve as reference tissue reflecting background activity concentration.

The peptide blocking dose (40 μg per mouse, 1.3 mg/kg) reduced the concentration of ⁶⁸Ga-PPF11N in the stomach to 0.19±0.02% ID/g in females and 0.32±0.07% ID/g in males.

As the route of ⁶⁸Ga-PPF11N elimination is believed to be through the renal system, high exposure in the kidney is expected. Moreover, the peptide blocking dose was unable to reduce kidney exposure, which demonstrates that kidney exposure is CCK2R-target independent and is most likely caused by elimination and/or renal uptake.

CCK2R is expressed in the stomach and consequently an accumulation over the time of ⁶⁸Ga-PPF11N is observed. At late time point (>60 minutes), stomach is the organ with the highest retention with 1.71±0.56% ID/g for females and 1.93±0.18% ID/g for males (excluding kidney which is the organ of elimination). Moreover, the peptide blocking dose was able to substantially reduce the stomach exposure in male and females demonstrating that the observed retention in stomach is dependent of the CCK2R expression.

The ⁶⁸Ga-PPF11N biodistribution broadly resembles that of ¹⁷⁷Lu-PPF11N published for 4 h post-injection in (Andreas Ritler et al., Elucidating the structure-activity relationship of the pentaglutamic acid sequence of minigastrin with the cholecystokinin receptor subtype 2, Bioconjugate Chem. 2019, 30, 3, 657-666; Alexander W. Sauter et al., Targeting of the Cholecystokinin-2 Receptor with the Minigastrin Analog 177Lu-DOTA-PP-F11N, J Nucl Med 2019; 60:393-399). Further, the above blocking experiment demonstrates that the binding of 68Ga-PPF11N to CCK2R receptors was specific. This blocking experiment also showed that gallium-68 radiolabeling achieved a sufficiently high specific activity of approx. 10 MBq/nmol for an ex vivo biodistribution study in mice with a non-blocking peptide mass dose of approx. 0.04 μg PPF11N (1.3 μg/kg).

This pre-clinical in vivo study demonstrates that the capacity of 68Ga-PPF11N and 177Lu-PPF11N to bind to CCK2R are comparable and, consequently, once injected into mice, the biodistribution profile of both molecules are very similar. We conclude from this study that 68Ga-PPF11N is a suitable diagnostic tool for specific CCKBR positive cancers and tumors. The imaging with 68Ga-PPF11N might be particular suited to pre-select patients who would benefit from a 177Lu-PPF11N treatment.

1-10. (canceled)

11. A labeled gastrin analogue represented by a formula:
⁶⁸Ga-Y-DGlu-DGlu-DGlu-DGlu-DGlu-DGlu-Ala-Tyr-Gly-Trp-Nle-Asp-Phe-NH₂,
wherein Y is a moiety chelating ⁶⁸Ga.

12. The labeled gastrin analogue according to claim 11, wherein Y is 1,4,7,10-tetraazacyclododecane-1,4,7,10-tetraacetic acid (DOTA), 1,4,7-triazacyclononane-1,4,7-triacetic acid (NOTA) or 1,4,7-triazacyclononane,1-glutaric acid-4,7-acetic acid (NODAGA).

13. The labeled gastrin analogue according to claim 11, wherein Y is DOTA.

14. The labeled gastrin analogue according to claim 11, wherein the labeled gastrin analogue is configured for use in a diagnostic method comprising steps of:
(i) administering the labeled gastrin analogue to a human patient who is to be diagnosed as to whether he or she suffers from a cholecystokinin B receptor (CCKBR) positive cancer or tumor; and
(ii) obtaining an image of body parts or tissue to be examined;
wherein the cancer or tumor is selected from small-cell lung cancer (SCLC), extrapulmonary small-cell carcinoma (EPSCC) and medullary thyroid cancer (MTC).

15. The labeled gastrin analogue according to claim 14, wherein:
the administration of the labeled gastrin analogue is used to identify patients that would benefit from a treatment with a compound of a formula:
¹⁷⁷Lu-DOTA-DGlu-DGlu-DGlu-DGlu-DGlu-DGlu-Ala-Tyr-Gly-Trp-Nle-Asp-Phe-NH₂,
wherein DOTA is 1,4,7,10-tetraazacyclododecane-1,4,7,10-tetraacetic acid.

16. A method for obtaining an image of a patient or body parts or tissue of the patient, the method comprising:
administering to a patient the labeled gastrin analogue defined in claim 11.

17. The method according to claim 16, which further comprises selecting PET (Positron Emission Tomography) as the method.

18. The method according to claim 16, which further comprises using the method to obtain an image of a cancer or tumor selected from small-cell lung cancer (SCLC), extrapulmonary small-cell carcinoma (EPSCC) and medullary thyroid cancer (MTC).

19. A kit comprising:
(a) a gastrin analogue represented by a formula:
Y-DGlu-DGlu-DGlu-DGlu-DGlu-DGlu-Ala-Tyr-Gly-Trp-Nle-Asp-Phe-NH₂,
wherein Y is a moiety capable of chelating ⁶⁸Ga; and
(b) excipients selected from a solvent or water, and one or more auxiliary substances or sodium acetate buffer, mannitol and ascorbic acid, said excipients being capable of dissolving the gastrin analogue and providing a solution capable of being labelled with ⁶⁸Ga in a chelation step, and said excipients being provided together or separately.

20. A method of using a kit, the method comprising:
using the kit according to claim 19 together with a ⁶⁸Ga generator to produce the labeled gastrin analogue represented by a formula:
⁶⁸Ga-Y-DGlu-DGlu-DGlu-DGlu-DGlu-DGlu-Ala-Tyr-Gly-Trp-Nle-Asp-Phe-NH₂,
wherein Y is a moiety chelating ⁶⁸Ga.
